(19) United States
(12) Patent Application Publication
(10) Pub. No.: US 2015/0284675 A1
(43) Pub. Date: Oct. 8, 2015

(54) STREPTOCOCCUS THERMOPHILUS STRAINS FOR TREATING HELICOBACTER PYLORI INFECTION

(71) Applicant: COMPAGNIE GERVAIS DANONE, Paris (FR)

(72) Inventors: Peggy Garault, Montlhery (FR); Raphaelle Bourdet-Sicard, Palaiseau (FR); Francis Megroud, Bordeaux (FR)

(21) Appl. No.: 14/438,458

(22) PCT Filed: Oct. 25, 2012
(86) PCT No.: PCT/IB2012/055883

§ 371 Date: Apr. 24, 2015

(51) Int. Cl. C12N 1/20; A23C 9/123; A61K 35/744

(52) U.S. Cl. CPC C12N 1/20; A61K 35/744; A23C 9/1238; A23Y 2240/75

(57) ABSTRACT

The present invention relates to a strain of Streptococcus thermophilus or a cell fraction thereof for use in the treatment or prevention of Helicobacter pylori infection.

The present invention relates to the field of probiotics. Particularly, the invention pertains to the use of a strain of Streptococcus thermophilus for the treatment or the prevention of Helicobacter pylori infection.

According to a definition recently approved by the National Yogurt Association (NYA) or the International Life Science Institute (ILSI) in the USA, probiotics are living micro-organisms which upon ingestion in a sufficient amount exert health benefits beyond basic nutrition. Probiotic bacteria have been described among species belonging to the genera Lactobacillus, Bifidobacterium, Streptococcus and Lactococcus, commonly used in the dairy industry. Probiotics are thought to intervene at the level of the gut microbiota by impeding the development of pathogenic microorganisms and/or by acting more directly on the immune system.

Helicobacter pylori (H. pylori) is a Gram-negative spiral-shaped bacteria that colonizes the human gastric mucus layer of more than 50% of the world's population. While the majority of individuals infected with H. pylori is asymptomatic although their gastric epithelium show sign of inflammation, 15% to 20% of H. pylori infected individuals develop diseases. Indeed, H. pylori is the major causative agent of chronic active gastritis, peptic ulcer diseases, atrophy, metaplasia, dysplasia, gastric cancer and gastric mucosa associated lymphoid tissue (MALT) lymphoma (see for review Fox and Wang, 2007 and Polk and Peek, 2010).

During infection, H. pylori binds specifically to gastric epithelial cells lining the gastric epithelium through several adhesion molecules (adhesins) produced by the bacteria, such as BabA and SabA proteins. Adhesion to the gastric epithelial cells protects the bacteria from liquid flow, peristaltic movement and shedding of the mucous layer. H. pylori adhesion to the gastric mucosa induces signal transduction pathways within the gastric epithelial cells, leading to gastric epithelial cell damages and atrophy via oxidative stress, apoptosis and/or autophagy mechanisms. Accordingly, H. pylori adhesion to gastric epithelial cells is a key step in the establishment of an infection of the gastric mucosa.

The standard treatment in patients infected with H. pylori is two antibiotics associated to a proton pump inhibitor (PPI), so called triple therapy. However, H. pylori eradication rate following triple therapy is dropping down because of antibiotic resistance or poor compliance. Further, despite several clinical trials, there is no effective vaccine available on the market yet.

It appears from the foregoing that there is a need for alternatives or complements to triple therapy for the treatment or for the prevention of H. pylori infection.

The use of probiotics has been proposed as alternatives or complements to triple therapy for treating or preventing H. pylori infection. For instance, Lactobacillus reuteri is considered as a candidate probiotic for inhibiting the growth of H. pylori since it produces the potent antimicrobial substance reuterin (3-hydroxypropionaldehyde) (International Application WO 2004/031368). Boyanova et al. (2009) have found several Lactobacillus delbrueckii subsp. bulgaricus strains that inhibit the growth of H. pylori strains in vitro. Simova et al. (2009) disclose a Lactobacillus delbrueckii strain (BB18) producing an inhibitory peptide (bacteriocin) and strongly inhibiting H. pylori. Linsalata et al. (2004) found that the Lactobacillus brevis strain CD2 is capable of reducing the intragastric H. pylori load, and suggested that it might be due to the elevated arginine deiminase activity of this strain, which would deprive H. pylori of arginine, and inhibit their growth and proliferation.

The inventors have found that the bacterial species Streptococcus thermophilus (S. thermophilus) is capable of decreasing the load of H. pylori strains in vivo.

Accordingly, a subject of the present invention is a Streptococcus thermophilus strain for use for treating or preventing Helicobacter pylori infection.

Said Streptococcus thermophilus can be used as a medicament, including a pharmaceutical composition and a functional food.

Said S. thermophilus strain is capable of decreasing the load of H. pylori strains in the stomach of a subject infected with H. pylori.

In a preferred embodiment, said S. thermophilus strain is the strain CNCM I-1520. This strain was deposited by the Applicant, according to the Budapest Treaty, at CNCM (Collection Nationale de Cultures de Microorganismes, 25 rue du Docteur Roux, Paris) on Dec. 30, 1994. This strain is disclosed in International Application WO 96/20607. This strain is also referred to as DN-001 147.

The present invention also encompasses mutant strains or genetically transformed strains derived from the parent strain CNCM I-1520, provided that they are capable of decreasing the load of H. pylori strains in the stomach of a subject infected with H. pylori. Method for assessing the capacity of a S. thermophilus strain to decrease the load of H. pylori strains in the stomach of a subject infected with H. pylori are described in the Examples below. These mutant or genetically transformed strains can be strains wherein one or more endogenous gene(s) of the parent strain CNCM I-1520 has (have) been mutated, for instance to modify some of their metabolic properties (e.g., their ability to ferment sugars, their resistance to acidity, their survival to transport in the gastrointestinal tract, their post-acidification properties or their metabolite production). They can also be strains resulting from the genetic transformation of the parent strain CNCM I-1520 by one or more gene(s) of interest, for instance in order to give to said genetically transformed strains additional physiological features, or to allow them to express proteins of therapeutic or vaccinal interest that one wishes to administer through said strains. These mutant or genetically transformed strains can be obtained from the parent strain CNCM I-1520 strain by means of the conventional techniques for random or site-directed mutagenesis and genetic transformation of Streptococcus, such as those described by Biswas et al., 1993 and Maguin et al., 1996, or by means of the technique known as “genome shuffling”, such as described by Yu et al., 2008.

A subject of the present invention is also a cell fraction which can be obtained from a S. thermophilus strain capable of decreasing the load of H. pylori strains in the stomach of a subject infected with H. pylori, preferably the strain CNCM I-1520, provided that said cell fraction is capable of decreasing the load of H. pylori strains in the stomach of a subject infected with H. pylori, for use for treating or preventing H. pylori infection. Said cell fraction is in particular DNA preparations or bacterial wall preparations obtained from cultures of said strain. It may also be culture supernatants or fractions of these strains. The cell fractions suitable for this use can be chosen, for example, by testing their properties on the load of H. pylori strains in the stomach of a subject infected with H pylori.

A subject of the present invention is also a composition comprising a Streptococcus thermophilus strain according to the present invention, preferably the strain CNCM I-1520, or a cell fraction according to the present invention, for use for treating or preventing H. pylori infection.

In the composition of the invention, said strain can be used in the form of whole bacteria which may be living or dead. Alternatively, said strain can be used in the form of a bacterial lysate. Preferably the bacterial cells are present as living, viable cells.

The composition of the invention can be in any form suitable for administration, in particular oral administration. This includes for instance solids, semi-solids, liquids, and powders. Liquid composition are generally preferred for easier administration, for instance as drinks.

The composition can comprise at least 10⁵ cfu, preferably at least 10⁶ cfu, per gram dry weight, of at least one bacterial strain as mentioned above.

The composition can further comprise other strains of Streptococcus thermophilus and/or other strains of bacteria than the strains according to the present invention, in particular probiotic strain(s), such as Lactobacillus, Bifidobacterium and Lactococcus strain(s).

In a preferred embodiment, the composition comprises the Streptococcus thermophilus strain CNCM I-1520, the Streptococcus thermophilus strain CNCM I-1521 (also referred to as DN-001 339) and the Lactobacillus bulgaricus strain CNCM I-1519 (also referred to as DN-100182), and optionally a Lactobacillus paracasei strain, preferably the Lactobacillus paracasei subsp. paracasei CMCM I-1518 (also referred to as DN-114 001). All these strains are described in International Application WO 96/20607.

When the bacteria are in the form of living bacteria, the composition may typically comprise 10⁵ to 10¹³ colony forming units (cfu), preferably at least 10⁶ cfu, more preferably at least 10⁷ cfu, still more preferably at least 10⁸ cfu, and most preferably at least 10⁹ cfu per gram dry weight of the composition. In the case of a liquid composition, this corresponds generally to 10⁴ to 10¹² colony forming units (cfu), preferably at least 10⁵ cfu, more preferably at least 10⁶ cfu, still more preferably at least 10⁷ cfu, and most preferably at least 10⁹ cfu/ml.

The composition can be a pharmaceutical composition or a nutritional composition, including food products, food supplements and functional food. More particularly, the composition can be a medicament, including a pharmaceutical composition and a functional food.

A “food supplement” designates a product made from compounds usually used in foodstuffs, but which is in the form of tablets, powder, capsules, potion or any other form usually not associated with aliments, and which has beneficial effects for one's health. A “functional food” is an aliment which also has beneficial effects for one's health. In particular, food supplements and functional food can have a physiological effect—protective or curative—against a disease, for example against a chronic disease.

The nutritional composition according to the invention also includes a baby food, an infant milk formula or an infant follow-on formula. Preferably the present composition is a nutraceutical or a pharmaceutical product, a nutritional supplement or medical food.

The composition can be a dairy product, preferably a fermented dairy product. The fermented product can be present in the form of a liquid or present in the form of a dry powder obtained by drying the fermented liquid. Examples of dairy products include fermented milk and/or fermented whey in set, stirred or drinkable form, cheese and yoghurt.

The fermented product can also be a fermented vegetable, such as fermented soy, cereals and/or fruits in set, stirred or drinkable forms.

In a preferred embodiment, the fermented product is a fresh product. A fresh product, which has not undergone severe heat treatment steps, has the advantage that the bacterial strains present are in the living form.

A subject of the present invention is also the use of a S. thermophilus strain as defined above, preferably the strain CNCM I-1520, or a composition as defined above for the manufacture of a medicament for treating or preventing H. pylori infection.

A subject of the present invention is also a method for treating or preventing H pylori infection in a subject in need thereof, said method comprising administering to said subject a therapeutically effective amount of a S. thermophilus strain as defined above, preferably the strain CNCM I-1520, or a composition as defined above.

Determination of a therapeutically effective amount is well known from the person skilled in the art, especially in view of the detailed disclosure provided herein.

A subject of the present invention is also a method for the manufacture of a medicament for treating or preventing H. pylori infection, said method comprising incorporating a S. thermophilus strain as defined above, preferably the strain CNCM I-1520, or a cell fraction as defined above, into at least one pharmaceutically acceptable diluent, carrier or excipient.

As used herein, the treatment or prevention encompasses inter alia: preventive infection and/or decreasing the load of H. pylori. The treatment or prevention also encompasses addressing at least one of the symptoms associated with H. pylori mentioned below.

Methods for diagnosing a H. pylori infection are known in the art. By way of example, diagnosis of a H. pylori infection can be made by checking by a blood antibody test, a stool antigen test or the carbon urea breath test. It can also be made by biopsy under endoscopy followed by an urease test, a histological examination, a microbial culture or a quantitative Real-Time PCR.

The symptoms or diseases associated with H. pylori infection are stomach ache, abdominal pain, regurgitation, vomiting, belching, flatulence, nausea, chronic active gastritis, peptic ulcer diseases, atrophy, metaplasia, dysplasia, gastric cancer and gastric mucosa associated lymphoid tissue (MALT) lymphoma.

The present invention will be understood more clearly from the further description which follows, which refers to examples illustrating the capacity of the S. thermophilus strain CNCM I-1520 of decreasing the load of H. pylori strains in vivo, as well as to the appended figures.

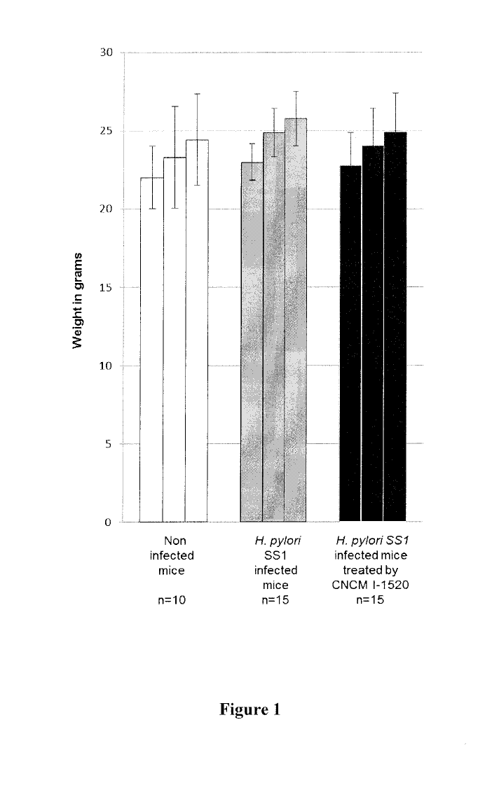

FIG. 1 shows the change in the weight (in grams) of non infected mice, mice infected with H. pylori SS1 receiving a control product, or infected with H. pylori SS1 and treated with S. thermophilus strain CNCM I-1520, measured just before the treatment (first bar), 3 weeks after the treatment (second bar) and just before sacrifice (third bar), obtained for 2 independent experiments.

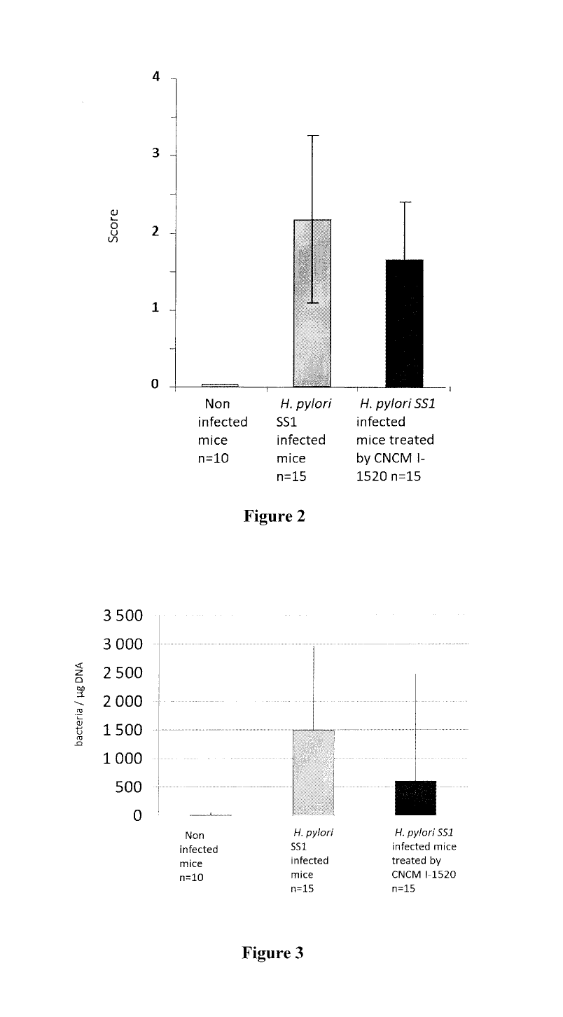

FIG. 2 shows the score of infection obtained by immunohistochemistry using anti-H. pylori antibodies in mice (i) non-infected with H. pylori, (ii) infected with H. pylori SS1 receiving a control product (non-fermented milk) and (iii) infected with H. pylori SS1 and treated with S. thermophilus CNCM I-1520. Definition of scores: 0: no infected gland, 1: rare infected glands, 2: 25% infected glands, 3: from 25 to 50% infected glands, 4: >50% infected glands.

FIG. 3 shows the quantification of H. pylori SS1 DNA obtained by Real-Time PCR in mice (i) non-infected with H. pylori, (ii) infected with H. pylori SS1 but receiving a control product (non-fermented milk) and (iii) infected with H. pylori SS1 and treated with S. thermophilus CNCM I-1520.

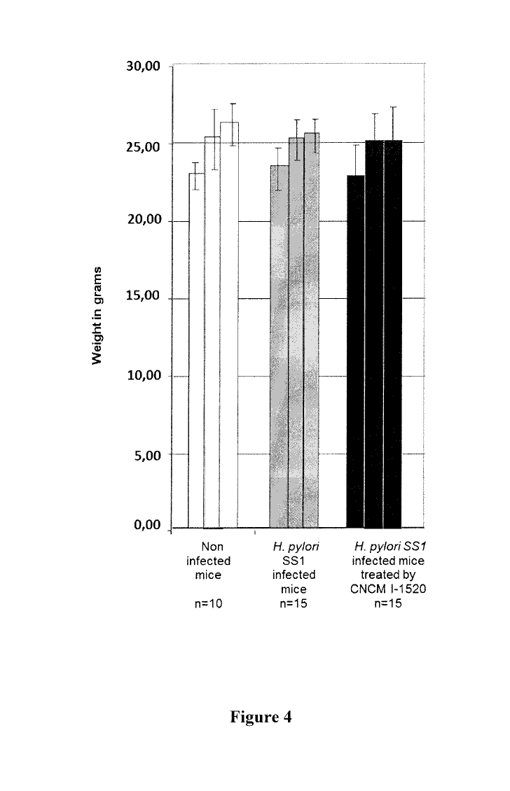

FIG. 4 shows the change in the weight (in grams) of non infected mice, mice infected with H. pylori SS1 receiving a control product, or infected with H. pylori SS1 and treated with S. thermophilus strain CNCM I-1520, measured just before the treatment (first bar), 3 weeks after the treatment (second bar) and just before sacrifice (third bar), obtained for 2 independent experiments.

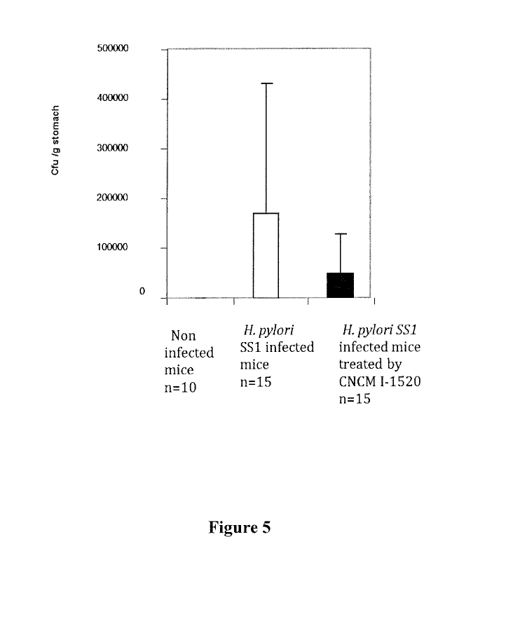

FIG. 5 shows the quantification of H. pylori SS1 obtained by bacterial culture on plate (in cfu (Colony Forming Unit) per gram of mice stomach) (i) non-infected with H pylori, (ii) infected with H. pylori SS1 but receiving a control product (non-fermented milk) and (iii) infected with H. pylori SS1 and treated with S. thermophilus CNCM I-1520.

### EXAMPLE 1

### Effect of the S. thermophilus Strain CNCM I-1520 on the Load of H. pylori in a Mice Model Determined by Histological and qRT-PCR Methods

### 1.1 Material & Methods

Helicobacter pylori

H. pylori strain SS1 having a very good colonization ability of mouse gastric mucosa (Lee et al., 1997) was used. Identity of the strain was checked by sequencing the genes glm, hspA and vacA (Raymond et al., 2004; Espinoza et al., 2011; Zhang et al., 2007).

Streptococcus thermophilus

Milk product fermented by S. thermophilus strain CNCM I-1520 was prepared as follows: First culture in M17 was prepared from frozen strain and incubated at 37° C. for 17 h. A second culture was prepared in skimmed milk enriched with yeast extract (2 g/L) by inoculation at 1% from the first culture and incubated at 37° C. for 17 h. A third culture was prepared in milk enriched with yeast extract (2 g/L) by inoculation at 1% from the second culture and incubation at 37° C. until pH 4.7 was reached. The product was finally prepared by inoculation of milk enriched with yeast extract (2 g/L) at 1% with the third culture until pH 4.8 was reached. Products were stored at −80° C. Bacterial count was carried out in M17 after 48 h incubation. Bacterial count was 1.5×10⁹ cfu/mL.

Mice

40 BALB/cBy/J female mice of 5 weeks old (Charles River, France) and tested as SPF (<<specific pathogen free>>) were split into groups: 2 groups of 15 mice were infected and 1 group of 10 mice was used as non infected control. Mice were fed with food poor in vitamins to enhance the lesion development induced by H. pylori.

Infection (8 Weeks)

6 weeks old mice received a hydric diet for 1 day and then were force-fed the following morning with 250 μL of an enriched suspension of the strain H. pylori SS1 (1 to 2 Petri dishes of H. pylori for 5 mice). The mice were put in a cage with a normal diet. Then, the mice received a hydric diet again in the evening. This protocol was repeated for 3 days.

Treatment (6 Weeks)

Eight weeks after their infection, mice were treated for 6 weeks with milk products containing S. thermophilus CNCM I-1520. 120 g of milk product were given per cage per day in feeding-bottles instead of water. The feeding-bottles were changed every day. To assess the quantity of products ingested per animal, the feeding-bottles were weighed. Further, mice were weighted just before the treatment, 3 weeks after the treatment and just before sacrifice (results are shown in FIG. 1).

Mice control groups received milk enriched with yeast extract (2 g/L) (i.e., without any S. thermophilus strain).

Sacrifices

Mice were sacrificed by cervical dislocation. Laparotomy was performed. Stomachs were isolated and gastric mucosa was washed in physiological serum.

Stomach was cut through the middle from the esophagus to the duodenum. For the right half stomach, cardia was eliminated, and then this half stomach was put in physiological serum to be used for the molecular study. The left half stomach was used for histology.

Histology

The left half stomach was fixed 1 night in 3.7% formol and washed with 70% ethanol and then paraffin-embedded and sectioned at 3 μm thickness.

Immunohistochemistry was carried out with an antibody anti-H. pylori antigens: primary antibody: anti-H. pylori (Dako, Ref. B0471); secondary antibody and DAB: Dako EnVision+ System-HRP (DAB) (Dako, Ref. K4011).

Molecular Study (qRT-PCR)

Right stomachs were homogenized (disrupted) in 0.2 ml physiological serum with a Potter-Elvehjem (the tube was weighted with and without the stomach tissue to know the weight of the tissue).

Total DNA was extracted from the crushed stomach with Arrow Stool DNA kit (NorDiag, Norway) following supplier recommendations. For each crushed stomach total DNA was resuspended in 180 μL TRIS buffer (10 mM).

Presence of DNA of H. pylori was quantified in DNA extracts by Real-Time PCR. Amplification was done with primers targeting 23S rRNA gene, present in two copies in H. pylori following the method described by Oleastro et al. (2003). For 20 μl of mix (MgCl₂ 25 mM, primers HPY-A et HPY-S 20 μM described by Ménard et al., 2002, sensor probe that is 5′ labeled with LC-Red 640 and 3′ phosphorylated and anchor probe that is 3′ labeled with fluorescein (both probes described by Oleastro et al. 2003) 20 μM, buffer containing the enzyme (10×, kit FastStart DNA Master Hybridization Probes, Roche Diagnostics), 5 μl DNA at 200 ng/μl was added to be amplified in Light Cycler ROCHE, using the following program:

| | | | | | |
|---|---|---|---|---|---|
| Denaturation: | 95° | C. | 10 | min | |
| Amplification: | 50 | cycles | | | 20° C./sec |
| | 95° | C. | 0 | sec | |
| | 60° | C. | 20 | s | |
| | 72° | C. | 12 | sec | |
| Fusion: | 95° | C. | 0 | sec | |
| | 38° | C. | 50 | sec | 20° C./sec |

1.2 Results

The scores of infection obtained by immunohistochemistry are shown in FIG. 2. These results show that administration of a milk product fermented with the S. thermophilus strain CNCM I-1520 to mice infected with H. pylori decreases (not significantly) the score of infection compared with the score obtained with the treatment with the milk control.

The results obtained by Real-Time PCR are shown in FIG. 3. These results show that, in mice, the treatment with the milk product fermented with the S. thermophilus strain CNCM I-1520 significantly decreases the load of H. pylori compared to the treatment with the milk control.

### EXAMPLE 2

### Effect of the S. Thermophilus Strain CNCM I-1520 on the Load of H. Pylori in a Mice Model Determined by Microbiological Method

2.1 Material & Methods

The material & methods for this experiment are the same as those described in Example 1 above regarding the H. pylori strain, the S. thermophilus strain CNCM I-1520, the mice, the infection, treatment and sacrifice of the mice, with the following exceptions:
the bacterial count of S. thermophilus strain CNCM I-1520 was 1.43.10⁹ cfu/mL;
the change in the weight of the treated mice is shown in FIG. 4;
only the right half stomach of the mice was used for the microbiology study.

Microbiology Study: Culture of H. pylori

The half stomachs were browed in 0.2 ml physiological serum with a Potter (tube is weighed with the liquid with and without stomach to deduce the exact weigh of tissue), 100 μL of dilutions (10⁻¹ to 10⁻⁴) were spread on Petri dish containing pylori medium GSSA (Glaxo Selective Supplement A (20 μg/ml bacitracin, 1.07 μg/ml nalidixic acid, 0.33 μg/ml polymyxin B, and 10 μg/ml vancomycin) enriched with 10% blood. Bacterial count was carried out at 37° C. after 5 to 7 days incubation under microaerobic condition. H. pylori was identified by phenotypic and biochemistry behaviors (morphology, urease and oxydase assays).

2.2 Results

The results obtained by microbiology for the S. thermophilus strain CNCM I-1520 are shown in FIG. 5. These results show that, in mice, the treatment with the milk product fermented with the strain CNCM I-1520 significantly decreases the load of H. pylori compared to the treatment with the milk control.

### REFERENCES

Biswas I. et al., J. Bacteriol. 1993; 175:3628-3635.
Boyanova L. et al., Lett Appl Microbiol. 2009; 48:579-84.
Espinoza M G C. et al., J. Clin. Microbiol. 2011; 49:1650-1652
Lee A. et al., Gastroenterology. 1997; 112:1386-97.
Linsalata M. et al., Helicobacter. 2004; 9:165-172.
Maguin E. et al., J. Bacteriol. 1996; 178:931-935.
Ménard A. et al., Antimicrob Agents Chemother. 2002; 46:1156-1157.
Oleastro M. et al., J Clin Microbiol. 2003; 41:397-402.
Raymond J. et al., Emerging Infection Diseases 2004; 10:1815-1821.
Simonava et al., J Appl Microbiol. 2009; 106:692-701.
Yu L. et al., J. Biotechnol. 2008; 134:154-159.
Zhang et al., World J. Gastroenterol., 2007; 13:845-850

1. A Streptococcus thermophilus strain for use for treating or preventing Helicobacter pylori infection.

2. The S. thermophilus strain according to claim 1, characterized in that said strain is capable of decreasing the load of H. pylori strains in the stomach of a subject infected with H. pylori.

3. The S. thermophilus strain according to claim 1, characterized in that it is the strain CNCM I-1520.

4-8. (canceled)

9. A cell fraction obtained from a S. thermophilus strain as defined in claim 1, wherein it is capable of decreasing the load of H. pylori strains in the stomach of a subject infected with H. pylori, for use for treating or preventing H. pylori infection.

10. A cell fraction obtained from a S. thermophilus strain as defined in claim 2, wherein it is capable of decreasing the load of H. pylori strains in the stomach of a subject infected with H. pylori, for use for treating or preventing H. pylori infection.

11. A cell fraction obtained from a S. thermophilus strain as defined in claim 3, wherein it is capable of decreasing the load of H. pylori strains in the stomach of a subject infected with H. pylori, for use for treating or preventing H. pylori infection.

12. A composition comprising a S. thermophilus strain as defined in claim 1 for use for treating or preventing H. pylori infection.

13. A composition comprising a cell fraction as defined in claim 9 for use for treating or preventing H. pylori infection.

14. The composition according to claim 12, characterized in that it comprises at least 10⁵ cfu per gram dry weight of the Streptococcus thermophilus strain.

15. The composition according to claim 12, characterized in that it comprises at least 10⁶ cfu per gram dry weight of the Streptococcus thermophilus strain.

16. The composition according to claim 12, characterized in that it is a nutritional composition.

17. The composition according to claim 13, characterized in that it is a nutritional composition.

18. The composition according to claim 16, characterized in that it is a dairy product.

19. The composition according to claim 17, characterized in that it is a dairy product.
